(19) United States
(12) Patent Application Publication
(10) Pub. No.: US 2022/0118257 A1
(43) Pub. Date: Apr. 21, 2022

(54) TREATING INFLAMMATORY DISORDERS BY STIMULATION OF THE CHOLINERGIC ANTI-INFLAMMATORY PATHWAY

(71) Applicant: The Feinstein Institutes for Medical Research, Manhasset, NY (US)

(72) Inventors: Jared M. HUSTON, New York, NY (US); Kevin J. TRACEY, Old Greenwich, CT (US)

(21) Appl. No.: 17/646,144

(22) Filed: Dec. 27, 2021

Related U.S. Application Data
(63) Continuation-in-part of application No. 12/259,208, filed on Oct. 27, 2008, now Pat. No. 11,207,518.
Application No. 12/259,208 is a continuation-in-part of application No. 11/318,075, filed on Dec. 22, 2005.
(60) Provisional application No. 60/639,332, filed on Dec. 27, 2004.

(51) Int. Cl. A61N 1/36

(52) U.S. Cl. CPC A61N 1/36114; A61N 1/36128

(57) ABSTRACT

Described herein are methods for treating a subject suffering from or at risk for a condition mediated by an inflammatory cytokine cascade, by electrically or mechanically stimulating vagus nerve activity in an amount sufficient to inhibit the inflammatory cytokine cascade.

### CROSS REFERENCE TO RELATED APPLICATIONS

This patent application is a continuation-in-part of U.S. patent application Ser. No. 12/259,208, filed on Oct. 27, 2008, which claims priority as a continuation-in-part of U.S. patent application Ser. No. 11/318,075, filed on Dec. 22, 2005, which claims priority to Provisional Patent Application No. 60/639,332, field on Dec. 27, 2004. This patent application also claims priority to U.S. Provisional Patent Application No. 60/982,681, filed on Oct. 25, 2007.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The methods, systems and devices described herein are directed to treatment of inflammatory disorders by appropriate and controlled stimulation of all or a portion of the cholinergic anti-inflammatory pathway.

### BACKGROUND OF THE INVENTION

Vertebrates achieve internal homeostasis during infection or injury by balancing the activities of pro-inflammatory and anti-inflammatory pathways. In many disease conditions, this internal homeostasis becomes out of balance. For example, endotoxin (lipopolysaccharide, LPS) produced by Gram-negative bacteria activates macrophages to release cytokines that are potentially lethal (Tracey, K. J. et al., Science, 234:470-74 (1986); Dinarello, C. A., FASEB J., 8:1314-25 (1994); Wang, H., et al., Science, 285:248-51 (1999); Nathan, C. F., J. Clin. Invest., 79:319-26 (1987)).

Inflammation and other deleterious conditions (such as septic shock caused by endotoxin exposure) are often induced by pro-inflammatory cytokines, such as tumor necrosis factor (TNF; also known as TNF-α or cachectin), interleukin (IL)-1α, IL-1β, IL-6, IL-8, IL-18, interferon-γ, platelet-activating factor (PAF), macrophage migration inhibitory factor (MIF), and other compounds. Certain other compounds, for example high mobility group protein 1 (HMG-1), are induced during various conditions such as sepsis and can also serve as pro-inflammatory cytokines. Pro-inflammatory cytokines contribute to various disorders, notably sepsis, through their release during an inflammatory cytokine cascade. Inflammatory cytokine cascades contribute to deleterious characteristics, including inflammation and apoptosis, of numerous disorders.

Although the pro-inflammatory cytokines typically aid in protecting against invasive pathogens, failure to resolve the initial infection can result in overproduction and spillage of cytokines into the systemic circulation, causing shock and lethal tissue injury. Counter-regulatory mediators, including glucocorticoids and interleukin-10, function to limit excessive pro-inflammatory responses. However, these humoral “anti-inflammatory” mechanisms can also predispose the host to secondary infections and increase overall morbidity and mortality.

It has recently been shown that the nervous system regulates systemic inflammation through an α7 nicotinic acetylcholine receptor (α7nAChR)-dependent vagus nerve pathway to the spleen, termed the cholinergic anti-inflammatory pathway (Huston J M, Ochani M, Rosas-Ballina M, et al., J Exp Med 2006; 203:1623-1628(2006)). This anti-inflammatory pathway may form an inflammatory reflex, including the vagus nerve, the splenic nerve, the hepatic nerve, the facial nerve, and the trigeminal nerve.

Electrical stimulation of the anti-inflammatory pathway (e.g., the vagus nerve or other portions of the anti-inflammatory reflex such as the splenic nerve, the hepatic nerve, etc.) inhibits pro-inflammatory cytokine production and prevents tissue injury in experimental models of systemic inflammation. Such stimulation fails to regulate pro-inflammatory responses in α7nAChR-deficient or splenectomized animals, or following interruption of the common celiac vagus branches, indicating that the cholinergic anti-inflammatory pathway requires specific molecular, organ, and neural components.

Pharmacologic α7nAChR agonists may mimic the effects of stimulation, and administration of these agents to mice with polymicrobial sepsis improves survival and attenuates production of high mobility group box 1 (HMGB1), a critical mediator of lethal sepsis. It is unclear, however, whether chronic vagus nerve stimulation can also improve survival during lethal sepsis.

Furthermore, the components of the cholinergic anti-inflammatory pathway, such as the vagus nerve, also subsume a number of other physiological roles and functions. In particular, the vagus nerve activation and/or inhibition is involved in regulating cardiac function (e.g., heart rate), larynx function, diaphragm activity (respiration), stomach (digestion) and includes both motor and sensory functions, as well as being implicated in brain activity including consciousness. Thus, the stimulation of any portion of the cholinergic anti-inflammatory pathway, and particularly the vagus nerve, must be performed specifically and precisely, in order to avoid disrupting or undesirably modifying any of these other functions.

Described herein are methods, systems and devices for specifically regulating the cholinergic anti-inflammatory pathway by precisely regulated and applied stimulation. In particular, described herein are methods, systems and devices for treating inflammation by electrical stimulation that are sufficient to inhibit inflammation without substantially affecting heart rate or other cardiac parameters, digestion, respiration, or other biological systems regulated by components of the inflammatory reflex such as the vagus nerve.

For example, the methods described herein provide methods for inhibition of pro-inflammatory cytokine production and protection against the long and short-term effects of the pro-inflammatory cytokines.

### SUMMARY OF THE INVENTION

In general, described herein are methods, systems and devices for treating cytokine-mediated inflammatory conditions. These methods, systems and devices typically stimulate all or a portion of a patient's inflammatory reflex (e.g., the vagus nerve), to inhibit inflammation, pro-inflammatory cytokine production or release, or the pro-inflammatory cytokine cascade. For example, the inhibitory stimulation described herein includes extremely low duty-cycle stimulation, such as stimulation of the vagus nerve at levels that do not provoke other non-immune, biological effects mediated by the vagus nerve (e.g., blood pressure, heart rate, etc.). Also described is effective repeated and/or chronic stimulation of the vagus nerve. For example, the effective extremely low-intensity vagus stimulation, and particularly chronic (or repeated) stimulation of the cholinergic anti-inflammatory pathway, may include low or extremely low duty-cycle (e.g., less than 1%, less than 0.01%, less than 0.001%, less than 0.0001%) stimulation of the vagus nerve to effectively inhibit or beneficially modulate inflammation or cytokine-mediated inflammatory conditions.

Using the methods and devices described herein, inflammatory disorders can be treated in a subject by electrically stimulating the cholinergic anti-inflammatory pathway, including, without limitation, the vagus nerve. Surprisingly, it has also been discovered that the parameters of an electrical signal sufficient to treat inflammatory disorders are significantly milder than the parameters previously shown to inhibit the inflammatory cytokine cascade. Thus, it has been discovered that inflammatory disorders can be treated by an electrical signal having its current or voltage significantly smaller than electrical signals previously shown to inhibit inflammation.

Described herein are methods of treating an inflammation in a patient. For example, the method may include the steps of: stimulating the patient's vagus nerve with an electrical signal, wherein the signal voltage is between about 0.01 Volt to 1 Volt, the pulse width is from 0.1 ms to 5 ms; the signal frequency is from 0.1 Hz to 30 Hz; signal on-time is from 1 second to 120 seconds; and waiting for an off-time of at least 2 hours before re-stimulating the patient's vagus nerve. In some variations, the method may also include the step of monitoring one or more non-inflammatory vagal efferent effects (e.g., heart rate, blood pressure, heart rate variability, digestive processes) and modifying the stimulation so that it does not affect such parameters.

In some variations, the stimulation is transcutaneous stimulation of the vagus nerve. For example, the stimulation may be transdermal. In some variations, the intensity of the stimulation (e.g., the voltage between 0.01 and 1 V) is determined at the nerve. Thus the voltage applied to the electrode(s) may be adjusted based on the attenuation through the tissue.

The off-time before the vagus nerve is re-stimulated may be from about 2 hours to about 48 hours. In general “off-time” is the time between “stimulation-on” periods or burst of pulses. For example, a burst of signals at 30 Hz for 120 seconds may be followed by an off-time of 2 or more hours. During the off-time, a controller for the system may be locked so that additional stimulation cannot be applied. In some variations, the off-time before the vagus nerve is re-stimulated is between about 16 and about 30 hours.

The method may also include the step of monitoring the effect of the stimulation on inflammation in the patient. For example, the level of a pro-inflammatory cytokine prior to stimulation may be determined, as well as the level of a pro-inflammatory cytokine during/after stimulation, and the levels may be compared. The intensity and/of frequency of stimulation may be adjusted based on the monitoring.

Also described herein are methods of treating inflammation in a patient, including the steps of: applying an electrode to a patient suffering from or at risk for a chronic inflammatory disorder; stimulating the patient's vagus nerve with an electrical signal from the electrode, wherein the signal voltage is from 0.01 Volt to 1 Volt, pulse width is from 0.1 ms to 5 ms; signal frequency is from 0.1 Hz to 30 Hz; signal on-time is from 1 second to 120 seconds; and waiting for an off-time of at least 2 hours before re-stimulating the patient's vagus nerve.

As mentioned above, in some variations, the method may be transcutaneous stimulation.

The chronic inflammatory disorder treated may be selected from the group consisting of: appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease.

The off-time before the vagus nerve is re-stimulated may be from between about 2 hours to about 48 hours. For example, the off-time before the vagus nerve is re-stimulated may be between about 16 and about 30 hours.

Also described herein are methods of treating inflammation in a patient including the steps of: implanting an electrode into a patient suffering from a chronic inflammatory disorder; stimulating the patient's vagus nerve with an electrical signal from the implanted electrode, wherein the signal voltage is from 0.01 Volt to 1 Volt, pulse width is from 0.1 ms to 5 ms; signal frequency is from 0.1 Hz to 30 Hz; signal on-time is from 1 second to 120 seconds; and waiting for an off-time of at least 2 hours before re-stimulating the patient's vagus nerve.

The chronic inflammatory disorder treated may be selected from the group consisting of: appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease.

### BRIEF DESCRIPTION OF THE DRAWINGS

FIG. 1 is a simplified block diagram of one variation of a stimulator (e.g., an electrical stimulus generator) suitable for applying electrical stimulation.

FIG. 2 shows one suitable location for vagus nerve stimulation (VNS) using an electric signal generator and electrodes implanted in the patient's body.

FIG. 3 is an illustrative idealized electrical output signal waveform that may be useful for clarifying relevant parameters of the stimulation signal.

FIG. 4 is a bar plot illustrating the effect of the vagal electrical stimulation on endotoxemia as measured by a reduced percentage of serum TNF level.

FIG. 5 is a plot showing the effect of electrical VNS on TNF production in LPS-challenged mice. Vertical axis indicates percent suppression of TNF, horizontal axis indicates hours elapsed between the VNS stimulation and LPS challenge. The deviation of TNF suppression substantially exceeds the period of applied VNS.

FIG. 6 is a plot that shows an arthritis score in rats as a function of a number of days post collagen immunization to induced arthritis (PCID). VNS stimulation started on day 13 and continued until day 20. The VNS stimulation inhibited the development of PCID.

FIG. 7 illustrates the effect of direct stimulation of the vagus nerve, showing attenuation of serum TNF concentrations. BALB/c mice received LPS (7.5 mg/kg, intraperitoneally) 5 minutes before either surgical dissection alone or increasing intensities of electrical vagus nerve stimulation. Blood was collected 2 hrs after endotoxin administration. Data are presented as mean±SEM (n=6-8 per group; **p<0.05).

FIG. 8 illustrates the anti-inflammatory and cardioinhibitory effects of vagus nerve stimulation (VNS), showing that the two are dissociable. Electrocardiograms were recorded for 5 minutes from BALB/c mice subjected to neck dissection (sham surgery), vagus nerve dissection, transcutaneous vagus nerve stimulation (300 seconds, 2 Hz) or increasing intensities of electrical vagus nerve stimulation (1 V, 5 Hz, 2 milliseconds; 5 V, 5 Hz, 2 milliseconds; 5 V, 30 Hz, 2 milliseconds). Changes in mean heart rate were compared with baseline. Data are presented as mean±SEM (n=6-8 per group; **p<0.05).

FIG. 9A shows that the effective duration of action of vagus nerve stimulation is between 48 and 72 hrs after VNS. BALB/c mice were subjected to electrical vagus nerve stimulation (30 seconds, 1 V, 30 Hz, 0.5 milliseconds) or neck dissection alone (control) on day 0 and were allowed to recover for 2, 24, 48, or 72 hrs before LPS challenge (7.5 mg/kg; intraperitoneally). Blood was collected 2 hrs after LPS administration for tumor necrosis factor (TNF) analysis. Data are presented as mean±SEM (n=6-8 per group; **p<0.05).

FIG. 9B illustrates the inhibition of TNF release from macrophages for 48 to 72 hrs by acetylcholine. Primary human macrophages were treated with acetylcholine (10 μM) and pyridostigmine bromide (1 mM) for 1 hr, were transferred to fresh media, and then received 4 hrs of LPS (10 ng/mL) stimulation beginning 2, 24, 48, or 72 hrs after acetylcholine exposure. Supernatants were collected and TNF was analyzed by enzyme-linked immunosorbent assay. Data are presented as mean±SEM of two separate experiments (**p<0.05).

FIG. 10A illustrates the beneficial effect of vagus nerve stimulation (VNS) on serum glucose concentrations in lethal endotoxemia. BALB/c mice were subjected to electrical vagus nerve stimulation (30 seconds, 1 V, 5 Hz, 2 milliseconds) following lipopolysaccharide (LPS) administration (7.5 mg/kg, intraperitoneally). Animals were euthanized after 2 hrs. Data are presented as mean±SEM (n=5-8 per group; *p<0.05 vs. control; **p<0.05 vs. LPS).

FIG. 10B illustrates the effects of vagus nerve stimulation versus dexamethasone (DEX) on splenocyte viability. Mice were subjected to electrical vagus nerve stimulation (30 seconds, 1 V, 5 Hz, 2 milliseconds) or dexamethasone (25 mg/kg, intraperitoneally) 24 hrs before LPS administration (7.5 mg/kg, intraperitoneally). Animals were euthanized 2 hrs after LPS, spleens were harvested, and viable splenocytes were measured. Data are presented as mean±SEM (n=6 per group; **p<0.05).

### DETAILED DESCRIPTION OF THE INVENTION

Described herein are devices, systems, and method for decreasing inflammation by appropriate stimulation of one or more components of the cholinergic anti-inflammatory pathway, such as the vagus nerve. In particular, methods of stimulating the vagus nerve to decrease or suppress the release of pro-inflammatory cytokines are described.

Direct (e.g. electrical or mechanical) stimulation of vagus nerve of a subject alleviates the symptoms of inflammatory disorders. As used herein, a “subject” is preferably a mammal, more preferably a human patient but can also be a companion animal (e.g., dog or cat), a farm animal (e.g., horse, cow, or sheep) or a laboratory animal (e.g., rat, mouse, or guinea pig). Preferable, the subject is human. As used herein, the term “vagus nerve” is used in its broadest sense, and may include any nerves that branch off from the main vagus nerve, as well as ganglions or postganglionic neurons that are connected to the vagus nerve. The vagus nerve is also known in the art as the parasympathetic nervous system and its branches, and the cholinergic nerve. The vagus nerve innervates principal organs including, the pharynx, the larynx, the esophagus, the heart, the lungs, the stomach, the pancreas, the spleen, the kidneys, the adrenal glands, the small and large intestine, the colon, and the liver. Stimulation can be accomplished by direct or indirect stimulation of the vagus nerve or an organ served by the vagus nerve.

In some variations, the stimulation is calibrated or controlled so that it does not substantially affect any target or efferent except those related to inflammation. For example, the stimulation provided to the vagus nerve may be below the threshold or frequency for affecting other vagus nerve-mediated effects, such as changes in heart rate, blood pressure, etc.

As used herein, “direct stimulation” of the vagus nerve may mean activating or stimulating the vagus nerve by non-pharmacological means such as electrical, mechanical (e.g., massage or vibration), heat or UV irradiation. Activation can be accomplished by direct or indirect stimulation of the vagus nerve or an organ served by the vagus nerve. The vagus nerve innervates principal organs including, the pharynx, the larynx, the esophagus, the heart, the lungs, the stomach, the pancreas, the spleen, the kidneys, the adrenal glands, the small and large intestine, the colon, and the liver.

The disclosed methods include stimulating the any portion of the cholinergic anti-inflammatory pathway, including the vagus. For example, the entire vagus nerve (i.e., both the afferent and efferent nerves) may be stimulated, or efferent nerve bundles may be isolated and then stimulated directly. The latter method can be accomplished by separating the afferent from the efferent fibers in an area of the nerve where both types of fibers are present. Alternatively, the efferent fibers are stimulated where no afferent fibers are present, for example close to the target organ served by the efferent fibers. The efferent fibers can also be activated by stimulating the target organ directly, e.g., electrically, thus stimulating the efferent fibers that serve that organ. In other embodiments, the ganglion or postganglionic neurons of the vagus nerve can be stimulated. The vagus nerve can also be cut and the distal end can be stimulated, thus only stimulating efferent vagus nerve fibers.

The cholinergic anti-inflammatory pathway (e.g., vagus nerve) can be stimulated by numerous methods including manually, mechanically, electrically or by electromagnetic radiation. Mechanical means of nerve stimulation include, without limitation, stimulation by needle (e.g., acupuncture). There is evidence that response to acupuncture may be at least partially mediated by the vagus nerve. For example, it has been shown that the response to electroacupuncture is attenuated after vagotomy (Noguchi et al, Jpn. J. Physiol. 46(1): 53-58 (1996)). Mechanical stimulation may also include nerve stimulation using ultrasound as described, for example in Norton, BioMedical Engineering 2(1): 6 (2003). Stimulation of the vagus nerve using heat or electromagnetic radiation includes, without limitation, applying heat or infrared, visible, ultraviolet, or other electromagnetic frequencies from an energy source. The vagus nerve may also be stimulated by magnetic stimulation; a description of magnetic nerve stimulation is provided in Hsu et al, IEEE Trans Biomed Eng 50(11): 1276-85 (2003). The entire teachings of these publications are incorporated herein by reference.

The site of stimulation of the cholinergic anti-inflammatory pathway (e.g., of the vagus nerve) may be in the cervical region (in the neck) and a region peripheral and distal of the cervical region including, supra-diaphragmatical or sub-diaphragmatical regions. Peripheral, distal locations include branches of the vagus nerve that innervate organs, including but not limited to, the spleen, the small intestine and the large intestine. The vagus nerve may also be stimulated endotracheally or transesophageally. Endotracheal or transesophageal vagal nerve stimulation may be accomplished using an endotracheal/esophageal nerve stimulator (described, for example, in U.S. Pat. No. 6,735,471, incorporated herein by reference in its entirety). The vagus nerve can be stimulated transesophageally using one or more esophageal electrodes (described, for example, in U.S. Pat. No. 5,571,150). The vagus nerve can also be stimulated using a transcutaneous nerve stimulator (as described for example in U.S. Pat. No. 6,721,603, incorporated herein by reference in its entirety) or a percutaneous nerve stimulator. In one embodiment, the vagus nerve is stimulated in the cervical region. In another embodiment, the vagus nerve is stimulated at a peripheral, distal location. In another embodiment, the vagus nerve is stimulated in the brain by the device.

According to one embodiment, the vagus nerve is stimulated by delivering an electrical signal generated by any suitable vagus nerve stimulators. For example, a commercial vagus nerve stimulator such as the Cyberonics NCP, or an electric probe can be used.

Examples of suitable vagus nerve stimulators are described, for example, in U.S. Pat. Nos. 4,702,254; 5,154,172; 5,231,988; 5,330,507; 6,473,644; 6,721,603; 6,735,471; and U.S. Pat. App. Pub. 2004/0193231. The teachings of all of these publications are incorporated herein by reference in their entirety.

The vagus nerve can be stimulated by means of either an implanted device or a device worn external to the patient's body, such as Cyberonics NCP device described in U.S. Pat. No. 5,231,988 or a Medtronic device described in U.S. Pat. No. 5,330,507. Both patents describe apparati for stimulating the right or left vagus nerve with continuous and/or phasic electrical signal.

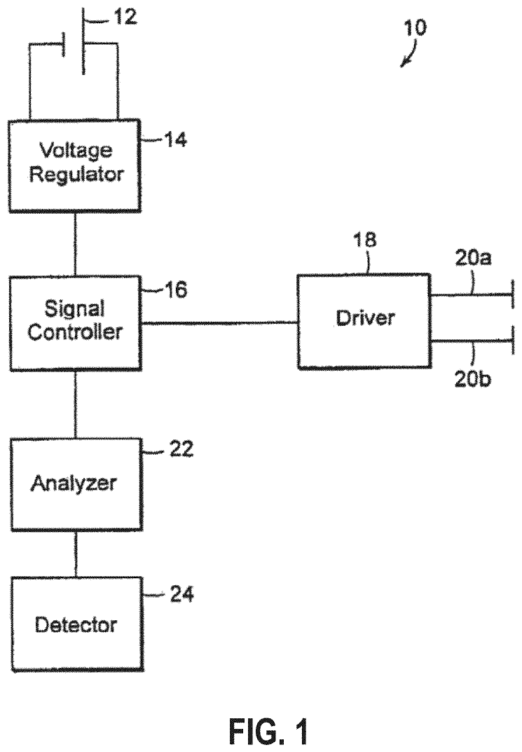

A schematic diagram of one variation of an electrical signal generator device suitable for practicing the methods described herein is shown in FIG. 1. Referring to FIG. 1, a typical signal generator 10 includes a battery (or set of batteries) 12, which may be of any type conventionally employed for powering medical electronic devices. Battery 12 is connected to a voltage regulator 14. Regulator 14 smoothes the battery output to produce steady output voltage as well as provides voltage multiplication or division if necessary.

Regulator 13 supplies power to signal controller 16. Signal controller 16 can includes a microprocessor. Signal controller 16 controls functions of the device such as output signal current or voltage, output signal frequency, output signal pulse width, output signal on-time, output signal off-time. Controller 16 can be programmed to control daily times for continuous or periodic modulation of vagal activity as well as output signal start delay time. Such programmability allows the output signal to be adjusted for the treatment regimen. The controller 16 may also limit the stimulation, so that it does not exceed a maximum level for intensity (e.g., voltage) or frequency (including on and/or off time, etc.). In some variations, the controller controls the stimulation by regulating the output to the electrodes (via Driver 18) so that the signal delivered to the tissue (e.g., vagus nerve) is within a predetermined range.

When device 10 is implanted, a built-in antenna (not shown) can be used to enable communication between device 10 and external programming or monitoring devices (not shown).

Signal controller 16 controls driver 18 which generates the desired electrical signal. The output signal is applied to the patient's body via electrodes 20a and 20b.

Analyzer 22 can be provided to process any relevant physiological parameters of a patient such as heart rate or blood pressure detected by detector 24.

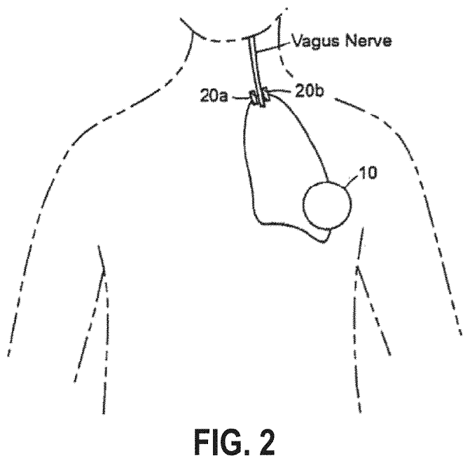

As mentioned above, device 10 can be worn external to the patient's body or can be implanted. FIG. 2 illustrates one embodiment of an implantable device for practicing the methods described herein, where signal generator 10 is implanted in the patient's chest in a pocket formed by the surgeon just below the skin. One suitable location for the generator is in the patient's chest, as a pacemaker pulse generator would be implanted, with the electrodes 20a and 20b implanted in the patient's neck.

Electrodes 20a and 20b can be bipolar stimulating electrodes of the type described in U.S. Pat. No. 4,573,481, incorporated herein by reference in its entirety. In this embodiment, electrodes form an assembly which is surgically implanted on the vagus nerve in the patient's neck. The two electrodes are wrapped around the vagus nerve, and the assembly is secured to the nerve by a spiral anchoring tether as disclosed in U.S. Pat. No. 4,979,511, incorporated herein by reference in its entirety.

Structurally, the electrode assembly can comprise two ribbons of platinum which are individually bonded to each of the two spiral loops wrapped around the vagus nerve. Each loop further includes silicone rubber. An additional helical loop that includes silicon rubber is provided to tether the electrode assembly to the vagus nerve. The inner diameter of the helical bipolar electrodes may typically be about two millimeters (mm), and an individual spiral is about seven mm long (measured along the axis of the nerve).

Instead of implanting the electrode assembly in the patient's neck, the assembly may be implanted on the vagus nerve as it innervates any of the organs listed above. The implantation of electrodes 20a and 20b is accomplished in substantially the same manner as was described for the neck location.

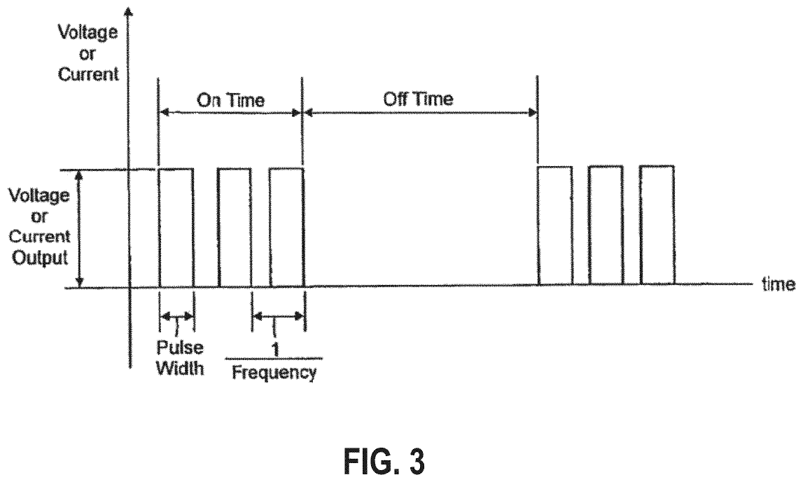

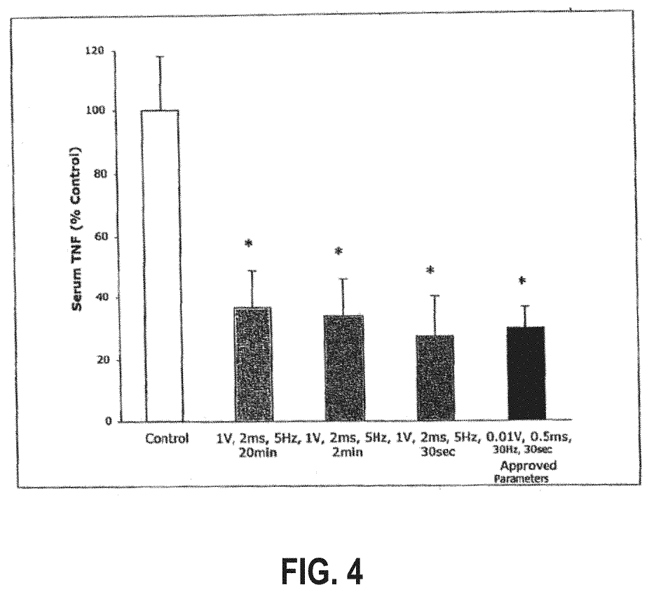

The operation of signal generator 10 to control and treat inflammatory disorders will be described by reference to the signal waveform and parameters shown in FIG. 3. The latter is an idealized representation of the output signal delivered by driver 18. FIG. 3 serves to clarify terminology used to refer to the parameters of an electrical signal. Such parameters include signal on-time, signal off-time, signal frequency, signal pulse width, signal current, and signal voltage. Treatment of inflammatory disorders can be accomplished by applying voltage to electrodes 20a and 20b as well as by driving a current between electrodes 20a and 20b. While the pulses shown in FIG. 3 have positive voltage or current output, electrical signals having negative outputs can also be used.

Signal controller 16 controls the output signal by limiting the output to a suitable range of parameters specified above with reference to FIG. 3. A range of each parameter can be chosen independently from any other parameter. Alternatively, a combination of ranges for any number of parameters can be chosen. Preferred examples of specific values for the parameters and combinations of parameters as provided below with respect to the controller are also applicable to the disclosed methods of treatment.

Signal controller can limit signal voltage to a range from about 0.01 Volt to about 1 Volt, preferably to a range from about 0.01 Volt to about 0.1 Volt, more preferably, to a range from about 0.01 Volt to about 0.05 Volt.

Signal controller can limit signal current to a range from about 1 mA to about 100 mA, preferably to a range from about 1 mA to about 10 mA, more preferably to a range from about 1 mA to about 5 mA.

In some embodiments, both signal voltage and signal current are controlled.

In other embodiments, either in addition to or independently from controlling signal voltage, signal current or both, signal controller can further control one or more parameters selected from pulse width, on-time and frequency. Signal controller can limit the pulse width to a range from about 0.1 ms to about 5 ms, preferably to a range from about 0.1 ms to about 1 ms, more preferably to a range from about 0.1 ms to about 0.5 ms. Signal controller can limit signal on-time from about 1 second to about 120 seconds, preferably, to a range of from about 10 seconds to about 60 seconds, more preferably, to a range from about 20 seconds to about 40 seconds. Signal controller can limit signal frequency to a range from about 0.1 Hz to about 30 Hz, preferably, to a range from about 1 Hz to about 30 Hz, more preferably, to a range from about 10 Hz to about 30 Hz.

Thus, the controllers for the implanted neurostimulators described herein may generally limit the applied charge. Specifically, since the controller may limit the current to between 1 mA and 100 mA (or specifically, 1 mA to about 10 mA, more preferably to a range from about 1 mA to about 5 mA), and the pulse width to a range from about 0.1 ms to about 5 ms (e.g., 0.1 ms to about 1 ms, more preferably to a range from about 0.1 ms to about 0.5 ms), and the signal frequency to a range from about 0.1 Hz to about 30 Hz (e.g., 1 Hz to about 30 Hz, more preferably, to a range from about 10 Hz to about 30 Hz); with a burst width (e.g., signal on-time) from about 1 second to about 120 seconds (10 seconds to about 60 seconds, more preferably about 20 seconds to about 40 seconds) every two hours or more (e.g., every two hours, every four hours, every 8 hours, every 12 hours, etc.), the controller may therefore limit the maximum charge delivered to be about (100 mA)×(5 msec)×(30 Hz)×(120 second) or 1.8 coulombs every two hours (or less). In some examples the controller may limit the maximum charge delivered by the implanted neurostimulator to be about 900 millicoulombs (e.g., 100 mA for 5 msec at 30 Hz and a 60 second burst width), or about 600 millicoulombs (e.g., 100 mA for 5 msec at 30 Hz and a 40 second burst width), or about 360 millicoulombs (e.g., 100 mA for 1 msec at 30 Hz and a 120 second burst width), or about 180 millicoulombs (e.g., 100 mA for 1 msec at 30 Hz and a 120 second burst width), or about 120 millicoulombs (e.g., 100 mA for 1 msec at 30 Hz and a 40 second burst width), or about 90 millicoulombs (e.g., 100 mA for 5 msec at 30 Hz and a 60 second burst width), or about 60 millicoulombs (e.g., 10 mA for 5 msec at 30 Hz and a 40 second burst width), or about 45 millicoulombs (e.g., 5 mA for 5 msec at 30 Hz and a 60 second burst width), or about 30 millicoulombs (e.g., 5 mA for 5 msec at 30 Hz and a 40 second burst width), or about 18 millicoulombs (e.g., 10 mA for 0.5 msec at 30 Hz and a 120 second burst width), or about 9 millicoulombs (e.g., 5 mA for 0.5 msec at 30 Hz and a 120 second burst width), or about 6 millicoulombs (e.g., 1 mA for 5 msec at 30 Hz and a 40 second burst width), or about 4.5 millicoulombs (e.g., 5 mA for 0.5 msec at 30 Hz and a 60 second burst width), or about 3 millicoulombs (e.g., 5 mA for 0.5 msec at 30 Hz and a 40 second burst width), or about 1.8 millicoulombs (e.g., 1 mA for 1 msec at 30 Hz and a 60 second burst width), or about 1.2 millicoulombs (e.g., 1 mA for 1 msec at 30 Hz and a 40 second burst width), or about 0.9 millicoulombs (e.g., 1 mA for 0.5 msec at 30 Hz and a 60 second burst width), or about 0.6 millicoulombs (e.g., 1 mA for 0.5 msec at 30 Hz and a 40 second burst width) every two hours.

Similarly, as described above, the controller for the implanted neurostimulator may provide a minimum amount of charge. For example, the controller for the implanted neurostimulators may apply a minimum of 10 nC every two hours (e.g., 1 mA for 0.1 msec at 0.1 Hz and a 40 second burst width).

The controller may be limited by, e.g., preventing the application of additional charge during this two hour (or longer) period, e.g., by locking the implanted neurostimulator so that additional charge is not delivered. As described herein, the controller may limit the charge delivered by limiting any one or more of the current, voltage, pulse width, frequency and burst width and off time.

In other embodiments, either in addition to or independently from controlling signal voltage and/or signal current, as well as signal width, signal frequency and/or signal on-time, signal controller can further control signal off-time. In one embodiment, a subject can be treated with one pulse. In another embodiment, signal controller can limit signal off-time to a range of over 5 minutes, preferably, over 2 hours, more preferably, over 4 hours, even more preferably, over 8 hours, still more preferably, over 12 hours. In another embodiment, signal controller can limit signal off-time to a range of from about 2 hours to about 48 hours, preferably to a range from about 4 hours to about 36 hours, more preferably, to a range from about 6 hours to about 36 hours. In other preferred embodiments, signal controller can limit signal off-time to a range selected from: from about 6 to about 36 hours, from about 12 to about 36 hours, from about 16 to about 30 hours and from about 20 to about 28 hours. Alternatively, signal off-time can be undefined as one skilled in the art will readily determine the desired time interval between two consecutive signals.

As mentioned above, various parameters can be limited to the specified ranges alone or in combination. In one example, signal controller can limit a combination of parameters as follows: signal voltage to a range from about 0.01 Volt to about 1 Volt; pulse width to a range from about 0.1 ms to about 5 ms; signal frequency to a range from about 0.1 Hz to about 30 Hz; signal on-time from about 1 second to about 120 seconds. Signal off-time can be undefined. Alternatively, signal off-time can be limited to a range over about 5 minutes. In other preferred embodiments, signal controller can limit signal off-time to a range selected from: from about 6 to about 36 hours, from about 12 to about 36 hours, from about 16 to about 30 hours and from about 20 to about 28 hours.

In another example, signal controller can limit a combination of parameters as follows: signal current to a range from about 1 mA to about 100 mA; pulse width to a range from about 0.1 ms to about 5 ms; signal frequency to a range from about 0.1 Hz to about 30 Hz; signal on-time from about 1 second to about 120 seconds. Signal off-time can be undefined. Alternatively, signal off-time can be limited to a range over about 5 minutes. In other preferred embodiments, signal controller can limit signal off-time to a range selected from: from about 6 to about 36 hours, from about 12 to about 36 hours, from about 16 to about 30 hours and from about 20 to about 28 hours.

In a preferred embodiment, signal controller can limit a combination of parameters as follows: signal voltage to a range from about 0.01 Volt to about 0.1 Volt; pulse width to a range from about 0.1 ms to about 1 ms; signal frequency to a range from about 1 Hz to about 30 Hz; signal on-time to a range of from about 10 seconds to about 60 seconds; signal off-time to a range of over 2 hours. Alternatively, signal off-time can be undefined. In other preferred embodiments, signal controller can limit signal off-time to a range selected from: from about 6 to about 36 hours, from about 12 to about 36 hours, from about 16 to about 30 hours and from about 20 to about 28 hours.

Alternatively, signal controller can limit a combination of parameters as follows: signal current to a range from about 1 mA to about 10 mA; pulse width to a range from about 0.1 ms to about 1 ms; signal frequency to a range from about 1 Hz to about 30 Hz; signal on-time to a range of from about 10 seconds to about 60 seconds; signal off-time to a range of over 2 hours. Alternatively, signal off-time can be undefined. In other preferred embodiments, signal controller can limit signal off-time to a range selected from: from about 6 to about 36 hours, from about 12 to about 36 hours, from about 16 to about 30 hours and from about 20 to about 28 hours.

More preferably, signal controller can limit a combination of parameters as follows: signal voltage to a range from about 0.01 Volt to about 0.05 Volt; pulse width to a range from about 0.1 ms to about 0.5 ms; signal to a range from about 10 Hz to about 30 Hz; signal on-time to a range from about 20 seconds to about 40 seconds; signal off-time to a range of from about 2 hours to about 24 hours. Alternatively, signal off-time can be undefined. In other preferred embodiments, signal controller can limit signal off-time to a range selected from: from about 6 to about 36 hours, from about 12 to about 36 hours, from about 16 to about 30 hours and from about 20 to about 28 hours. In other preferred embodiments, signal controller can limit signal off-time to a range selected from: from about 6 to about 36 hours, from about 12 to about 36 hours, from about 16 to about 30 hours and from about 20 to about 28 hours.

Alternatively, signal controller can limit a combination of parameters as follows: signal current to a range from about 1 mA to about 5 mA; pulse width to a range from about 0.1 ms to about 0.5 ms; signal to a range from about 10 Hz to about 30 Hz; signal on-time to a range from about 20 seconds to about 40 seconds; signal off-time to a range of from about 2 hours to about 24 hours. Alternatively, signal off-time can be undefined. In other preferred embodiments, signal controller can limit signal off-time to a range selected from: from about 6 to about 36 hours, from about 12 to about 36 hours, from about 16 to about 30 hours and from about 20 to about 28 hours.

As used herein, “treatment” may include prophylactic and therapeutic treatment. “Prophylactic treatment” refers to treatment before onset of an inflammatory condition to prevent, inhibit or reduce its occurrence. Therapeutic treatment is treatment of a subject who is already experiencing an inflammatory disorder.

“Inflammatory disorders” are usually mediated by an inflammatory cytokine cascade, defined herein as an in vivo release from cells of at least one pro-inflammatory cytokine in a subject, wherein the cytokine release affects a physiological condition of the subject. Non-limiting examples of cells that produce pro-inflammatory cytokines are monocytes, macrophages, neutrophils, epithelial cells, osteoblasts, fibroblasts, smooth muscle cells, and neurons.

As used herein, a “cytokine” is a soluble protein or peptide which is naturally produced by mammalian cells, and which act in vivo as humoral regulators at micro- to picomolar concentrations. Cytokines can, either under normal or pathological conditions, modulate the functional activities of individual cells and tissues. A pro-inflammatory cytokine is a cytokine that is capable of causing any of the physiological reactions associated with inflammation, such as: vasodialation, hyperemia, increased permeability of vessels with associated edema, accumulation of granulocytes and mononuclear phagocytes, or deposition of fibrin. In some cases, the pro-inflammatory cytokine can also cause apoptosis, such as in chronic heart failure, where TNF has been shown to stimulate cardiomyocyte apoptosis. Non-limiting examples of pro-inflammatory cytokines are tumor necrosis factor (TNF), interleukin (IL)-1.alpha., IL-1.beta., IL-6, IL-8, IL-18, interferon-γ, HMG-1, platelet-activating factor (PAF), and macrophage migration inhibitory factor (MIF). In one embodiments, the pro-inflammatory cytokine that is inhibited by cholinergic agonist treatment is TNF, an IL-1, IL-6 or IL-18, because these cytokines are produced by macrophages and mediate deleterious conditions for many important disorders, for example endotoxic shock, asthma, rheumatoid arthritis, inflammatory bile disease, heart failure, and allograft rejection. In most preferred embodiments, the pro-inflammatory cytokine is TNF.

Pro-inflammatory cytokines are to be distinguished from anti-inflammatory cytokines, such as IL-4, IL-10, and IL-13. In preferred embodiments, release of anti-inflammatory cytokines is not inhibited by cholinergic agonists.

When referring to the effect of the vagus nerve stimulation on an inflammatory disorder, the use of the terms “treatment”, “inhibition”, “decrease” or “attenuation” encompasses at least a small but measurable reduction in the symptoms associated with the disorder being treated.

“Treatment” includes both therapeutic and prophylactic treatments.

Described herein are methods of treatment of inflammatory disorders or conditions mediated by an inflammatory cytokine cascade. In one aspect, the disorder is not ileus, asthma or cystic fibrosis.

In another embodiment, the methods described herein may be methods of treating ileus. As used herein, “ileus” means a short term cessation (less than one month, typically, less than 2 weeks, often less than 1 week) of function of bowels not caused by chronic condition such as gastric ulcer, gastroesophageal reflux, diabetic gastroparesis, postvagotomy, and postgastrectomy. In one embodiment ileus is characterized by inflammation of intestinal smooth muscles.

The methods describe herein can be used to treat ileus caused by manipulation of the bowels during abdominal surgery (“post-operative ileus”), or administration of narcotics or chemotherapeutic agents such as during cancer chemotherapy. Successful treatment of ileus includes reduction and alleviation of symptoms of ileus. The terms “reduction” or “alleviation”, when referring to symptoms of ileus in a subject, encompass reduction in measurable indicia over non-treated controls. Such measurable indicia include, but are not limited to retention time of gastric content after gavage and myeloperoxidase activity (units per gram) in the gastrointestinal musculature. In preferred embodiments, the measurable indicia are reduced by at least 20% over non-treated controls; in more preferred embodiments, the reduction is at least 70%; and in still more preferred embodiments, the reduction is at least 80%. In a most preferred embodiment, the symptoms of ileus are substantially eliminated.

In one embodiment, the ileus to be treated is a post-operative ileus, i.e. ileus that occurs after abdominal surgery.

With respect to ileus, “treatment” includes pre-operative, peri-operative and post-operative treatment of ileus. Thus, “treatment” means prophylactic treatment of subjects at risk for ileus, for example, a subject undergoing abdominal surgery, experiencing abdominal surgery, or being administered narcotics or chemotherapeutic agents. With respect to ileus, “prophylactic treatment” refers to treatment before onset of ileus to prevent, inhibit or reduce the occurrence of ileus. For example, a subject at risk for ileus, such as a subject undergoing abdominal surgery, or about to undergo abdominal surgery, or being (or about to be) administered narcotics or chemotherapeutic agents can be prophylactically treated according to the method described herein prior to the anticipated onset of ileus. For example, a subject about to undergo surgery can be treated up to eight days before surgery, up to seven days before surgery, up to six days before surgery, up to five days before surgery, up to four days before surgery, up to three days before surgery, 48 hours prior to surgery, up to 36 hours prior to surgery, up to 24 hours prior to surgery, up to 12 hours prior to surgery, up to 6 hours before surgery, up to 3 hours before surgery, up to 2 hours before surgery, up to one hour before surgery and up to the onset of surgery. In another example, a subject can be treated during the surgery or administration of narcotics or chemotherapeutic agents. In another embodiment, the subject can be treated after the completion of surgery of administration of narcotics or chemotherapeutic agents. For example, a subject can be treated immediately after surgery, up to one hour after surgery, up to 2 hours after surgery, up to 3 hours after surgery, up to 6 hours after surgery, up to 12 hours after, up to 24 hours after, up to 36 hours after, up to 48 hours after surgery, up to three days after surgery, up to four days after surgery, up to five days after surgery, up to six days after surgery, up to seven days after surgery or up to eight days after surgery. “Treatment” of ileus also includes therapeutic treatment, where the subject is already experiencing ileus.

In one example, the subject can be treated pre-operatively, post-operatively, or peri-operatively once, twice, three times, four times or more than four times during the intervals described above. Alternatively, the subject can be treated by any combination of pre-operative, post-operative or peri-operative regimens during the intervals described above.

Preferably, ileus is treated by stimulating the vagus nerve endotracheally or transesophageally. Any device capable of performing this function can be employed. An example of an endotracheal/esophageal nerve stimulator is described in U.S. Pat. No. 6,735,471, incorporated herein by reference in its entirety.

Also described herein is the use of any of the devices described above in the manufacture of a therapeutic article for treating inflammatory disorders in a subject, wherein the device, in operation, directly or indirectly stimulates the vagus nerve to treat inflammatory disorders. The term “in operation” is intended to mean the device during use or application of the device on, to, or near the subject to directly stimulate the vagus nerve to treat inflammatory disorders.

In a further aspect, the methods described relate to the use of a device in the manufacture of a therapeutic article for treating inflammatory disorders in a subject, wherein the device is used solely to stimulate the vagus nerve for the purpose of treating inflammatory disorders. The term “solely” includes the use of the device to selectively treat inflammatory disorders where other diseases or conditions could potentially be treated by stimulation of the vagus nerve.

In some variations, only inflammatory disorders are treated or affected by the direct stimulation of the vagus nerve by the device. In one embodiment, the device may be adapted specifically to treat only inflammatory disorders by direct stimulation of the vagus nerve.

The condition can be one where the inflammatory cytokine cascade causes a systemic reaction, such as with septic shock. Alternatively, the condition can be mediated by a localized inflammatory cytokine cascade, as in rheumatoid arthritis.

Non-limiting examples of conditions which can be usefully treated using these methods include ileus, appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease.

In another embodiment, the examples of conditions which can be usefully treated include appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease.

In more preferred embodiments, the condition is ileus, appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection or graft-versus-host disease. In more preferred embodiments, the condition is endotoxic shock.

In another embodiment, the condition is appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection or graft-versus-host disease.

In another preferred embodiment, the conditions are ileus, sepsis, endotoxic shock, allograft rejection, rheumatoid arthritis, adult respiratory distress syndrome, asthma, systemic lupus erythematosis, pancreatitis, peritonitis, burns, myocardial ischemia, allograft rejection, graft-versus-host disease, congestive heart failure, organ ischemia, reperfusion injury, cachexia and cystic fibrosis.

In another embodiment, the conditions are sepsis, endotoxic shock, allograft rejection, rheumatoid arthritis, adult respiratory distress syndrome, asthma, systemic lupus erythematosis, pancreatitis, peritonitis, burns, myocardial ischemia, allograft rejection, graft-versus-host disease, congestive heart failure, organ ischemia, reperfusion injury, cachexia and cystic fibrosis.

In another preferred embodiment, the conditions are ileus, appendicitis, ulcerative colitis, Crohn's disease, allergy, reperfusion injury, systemic lupus erythematosus, hepatitis, Behcet's syndrome, multiple sclerosis and atherosclerosis.

In another embodiment, the conditions are appendicitis, ulcerative colitis, Crohn's disease, allergy, reperfusion injury, systemic lupus erythematosus, hepatitis, Behcet's syndrome, multiple sclerosis and atherosclerosis.

In another embodiment, the conditions are ileus, endotoxic shock and sepsis. In another embodiment, the conditions are endotoxic shock and sepsis.

Also described herein is the use of an electrical signal generator for construction of a medical device for treating a subject suffering from, or at risk for ileus. In yet another embodiment, an electrical signal generator may be used for construction of a medical device for treating a subject suffering from, or at risk for post-operative ileus. The device comprises an electrode assembly for delivering an electrical signal to the vagus nerve of the subject; and a controller controlling the electrical signal by limiting the signal voltage to a range from 0.01 Volt to 1 Volt. Preferably, the controller is limiting the signal voltage to a range from 0.01 Volt to 0.05 Volt. In another embodiment, the controller is limiting pulse width to a range from 0.1 ms to 5 ms; signal frequency to a range from 0.1 Hz to 30 Hz; and signal on-time to a range from 1 second to 120 seconds. In yet another embodiment, the controller is limiting signal voltage to a range from 0.01 Volt to 0.05 Volt; pulse width to a range from 0.1 ms to 0.5 ms; signal frequency to a range from 10 Hz to 30 Hz; and signal on-time to a range from 20 seconds to 40 seconds.

In another embodiment, an electrical signal generator may be used for construction of a medical device for treating a subject suffering from, or at risk for ileus. The device comprises an electrode assembly for delivering an electrical signal to the vagus nerve of the subject; and a controller controlling the electrical signal by limiting the signal voltage to a range from 0.01 Volt to 1 Volt, pulse width to a range from 0.1 ms to 5 ms; signal frequency to a range from 0.1 Hz to 30 Hz; signal on-time to a range from 1 second to 120 seconds; and signal off-time to a range over 2 hours.

Preferably, the controller limits the signal off-time to a range from 2 hours to 48 hours, and more preferably to a range of 16 hours to 30 hours. In another embodiment, the device, in operation, directly stimulates the vagus nerve to treat ileus. In another embodiment, the device is used solely to stimulate the vagus nerve for the purpose of treating ileus.

The methods and systems described above are illustrated by the following examples which are not intended to be limiting in any way.

### EXEMPLIFICATION

### Example 1: Electrical Vagus Nerve Stimulation Using Decreased Stimulation Intensities and Durations are Sufficient for Activation of the Cholinergic Anti-Inflammatory Pathway

To determine whether decreased stimulation parameters could achieve anti-inflammatory effects, intact vagus nerves were electrically stimulated at progressively lower stimulation intensities and durations in the setting of lethal endotoxemia. Male 8- to 12-week-old BALB/c mice (25-30 g; Taconic) were housed at 25° C. on a 12 hour light/dark cycle. Animals were allowed to acclimate to the facility for at least 7 days prior to experimental manipulation. Standard mouse chow and water were freely available. All animal experiments were performed in accordance with the National Institutes of Health (NIH) Guidelines under protocols approved by the Institutional Animal Care and Use Committee of the North Shore-Long Island Jewish Research Institute.

Mice were anesthetized with isoflurane (1.5-2.0%) and placed supine on the operating table. A ventral cervical midline incision was used to expose and isolate the left cervical vagus nerve. For electrical stimulation, the intact vagus nerve was placed across bipolar platinum electrodes (Plastics One) connected to a stimulation module (STM100C, Biopac Systems) and controlled by an acquisition system (MP150, Biopac Systems). Electrical stimulation parameters were programmed using AcqKnowledge software (Biopac Systems). Stimulation parameters included (100 mA, 2 ms, 5 Hz) for 20 min (10 min before LPS administration and 10 min after), (100 mA, 2 ms, 5 Hz) for 2 min (1 min before LPS administration and 1 min after), (100 mA, 2 ms, 5 Hz) for 30 sec (5 min after LPS administration), and (1 mA, 0.5 ms, 30 Hz) for 30 sec (5 min after LPS administration). Sham operated electrical VNS mice underwent cervical incision followed by dissection of the underlying submandibular salivary glands only. The vagus nerve was neither exposed nor isolated.

Mice were injected with endotoxin (Escherichia coli LPS 0111:B4; Sigma) that was dissolved in sterile, pyrogen-free saline at stock concentrations of 1 mg/ml. LPS solutions were sonicated for 30 min immediately before use for each experiment. Mice received an LD.sub.50 dose of LPS (7.5 mg/kg, i.p.). Blood was collected 2 h after LPS administration, allowed to clot for 2 h at room temperature, and then centrifuged for 15 min at 2,000.times.g. Serum samples were stored at −20.degree. C. before analysis. TNF concentrations in mouse serum were measured by ELISA (R & D Systems).

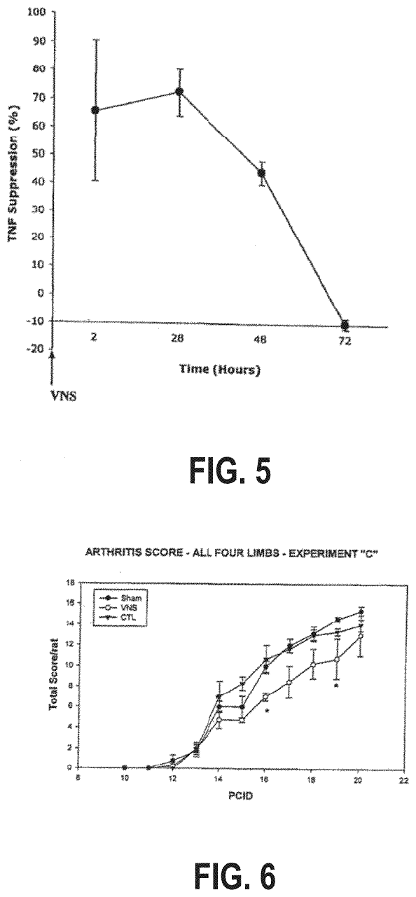

As shown in FIG. 5, all four stimulation parameters were sufficient for TNF suppression. The control mice group who received LPS followed by sham VNS had a mean serum TNF level of 2755.+−.424 pg/ml. Serum TNF levels in the electrical VNS groups were as follows; 20 min (712.+−.128 pg/ml, 25.8% of control, p=0.02), 2 min (688.+−.114 pg/ml, 25.0% of control, p=0.02), 30 sec at 100 mA (821.+−.378 pg/ml, 29.8% of control, p=0.46), and 30 sec at 1 mA (767.+−.144 pg/ml, 27.8% of control, p=0.03). The 30 sec 1 mA group corresponds to a clinically approved stimulation protocol (REF).

These results indicate that cholinergic anti-inflammatory pathway activation is responsive to physiologic, clinically well-tolerated electrical stimulation parameters. Additionally, the application of supraphysiologic current doses or prolonged stimulation durations does not provide any additional benefits in terms of reduced pro-inflammatory cytokine production.

### Example 2: The Effective Duration of Action of Electrical Vagus Nerve Stimulation-Mediated TNF Suppression is Between Two and Three Days

To determine how long the anti-inflammatory effects of vagus nerve stimulation last after the completion of stimulation, mice underwent electrical stimulation for 30 sec (1 mA, 0.5 ms, 30 Hz), and were allowed to recover for defined time periods prior to LPS administration. Control mice underwent sham surgery at time 0 and were challenged with LPS at the identical time periods as stimulated mice. Results for the four experimental groups are depicted in FIG. 5. Waiting for two hours between VNS and subsequent lipopolysaccharide (LPS) administration resulted in a 71% suppression of TNF (control=1606.+−.326 pg/ml vs. VNS=474.+−.157 pg/ml, p=0.01). Waiting for one day between VNS and LPS administration resulted in a 72% suppression of TNF (control=2813.+−.503 pg/ml vs. VNS=783.+−.87 pg/ml, p=0.004). Waiting two days between VNS and LPS resulted in a 44% suppression of TNF (control=1590.+−.351 pg/ml vs. VNS=892.+−.85 pg/ml, p=0.09). Finally, waiting for three days resulted in no TNF suppression (control=1253.+−.202 pg/ml vs. VNS=1393.+−.263 pg/ml, p=0.7). Animals were euthanized two hours after LPS administration.

These results indicate that the cholinergic anti-inflammatory pathway's effects are very long lasting, persisting for at least two days after stimulation. Furthermore, there was no significant difference in the anti-inflammatory effects between the two hour delay as opposed to a one day delay prior to LPS challenge. Finally, the data indicate that the anti-inflammatory effects of vagus nerve stimulation in this model had dissipated by three days after stimulation.

### Example 3: Electrical Vagus Nerve Stimulation Improves Severity of Arthritis in a Rat Model of Collagen-Induced Arthritis

To determine if vagus nerve stimulation could delay the onset or ameliorate the severity of arthritis in a rat collagen-induced arthritis model, rats received repeated vagus nerve stimulation via implanted electrodes for several days after collagen immunization and were scored for arthritis severity.

Purified Rat Type II Collagen (CII) (Chondrex, Redmond, Wash., USA) was dissolved in 0.01M acetic acid. Equal volumes of collagen solution and incomplete Freund's adjuvant (IFA; Difco Laboratories, Detroit, Mich.) were emulsified at 4.degree. C. so that 200 ul of emulsion contains 150 ug of rat CII (Akerlund et al, Clinical & Experimental Immunology 1999 115: 32-41; Kokkola R et al., Arthritis Rheum. 2003 48:2052-8.). Rats were immunized intradermally at the base of the tail with a volume of 200 ul per animal. A chronic, destructive arthritis developed with a mean onset of 14 days after immunization.

A previously described, arthritis clinical scoring system was utilized (Kokkola R et al., Arthritis Rheum 2003. 48(7): 2052-2058). This scoring system has proven reliable and highly discriminative for therapeutic studies (Akerlund et al, Clin Exp Immunol 1999, 115:32-41). Rats were observed daily for clinical signs of arthritis, including erythema and swelling of the joints. The interphalangeal joints of the digits, the metacarpophalangeal joint and wrist in the forepaw, and the metatarsophalangeal joint and ankle joint in the hind paw are each considered as one category of joint. Each paw was scored on a scale of 0-4 as follows: 0=unaffected, 1=1 type of joint affected, 2=2 types of joints affected, 3=3 types of joints affected, 4=3 types of joints affected and maximal erythema and swelling. An arthritis index was calculated for each rat and expressed as the cumulative score for all paws, with a maximum possible score of 16. Two independent observers performed all arthritis evaluations. The observers were additionally blinded to the identity of the animals.

Electrical vagus nerve stimulation was started on the 13th day post collagen immunization day (PCID). VNS rats were stimulated for 10 min once a day (5 V, 1-2 mA; 0.5 millisecond pulse; 30 Hz; 10 min on-time of alternating 30 seconds “on” and 300 seconds “off”) through day 20 (day 16 was skipped). These stimuli were generated using the STMISOC stimulation adapter, STM100C stimulator module, and MP150 Data Acquisition System, all from Biopac Systems, Inc. Where indicated, all animals were anesthetized using isoflurane inhalation gas (2-4%). During surgical procedures, animals were placed on a maintenance anesthesia dose via a mask delivery system. Following isoflurane anesthesia induction, animals were placed in supine position, and a 2 cm ventral midline cervical incision was made between the mandible and sternum. The subcutaneous tissue was dissected and retracted laterally. The mandible salivary glands were bluntly separated and retracted laterally. The left vagus nerve was isolated between the sternomastoid and sternohyoid muscles, dissected free from the neighboring carotid artery, and controlled with a 4-0 silk suture. A Teflon-coated silver electrode 0.003 inch in diameter was secured to the vagus nerve by multiple 360 degree circular wrappings around the nerve. The Teflon only was stripped from the ends of the wire to minimize electrical stimulation of the surrounding cervical muscles. The silver wire ends then were tunneled subcutaneously around the left neck to the dorsal cervical midline. At this point, they were exited through the skin and be attached to stimulating wires traveling through the tether apparatus.

As shown in FIG. 6, repeated electrical stimulation of the vagus nerve resulted in decreased clinical signs of arthritis (as measured by erythema and swelling of the joints) as compared with control and sham animals (stimulated rats: N=4; sham: N=5; control: N=3.) On day 16, the arthritis score in rats receiving vagus nerve stimulation was significantly less than that in control and sham animals (p<0.05). On day 19, the arthritis score in rats receiving vagus nerve stimulation was significantly less than that in sham treated animals (p<0.05). These results indicate that repeated vagus nerve stimulation is effective at lessening arthritis severity in collagen-induced arthritis.

### Example 4: Mouse Model for Electrical Stimulation with Endotoxemia

Animals. Male 8- to 12-wk-old BALB/c mice (25-30 g; Taconic) were housed in groups at 25° C. on a 12-hr light/dark cycle. Animals were acclimated to our facility for 7 or more days. All animal experiments were performed in accordance with the National Institutes of Health Guidelines under protocols approved by the Institutional Animal Care and Use Committee of the Feinstein Institute for Medical Research. Animals received different treatments as described subsequently. The allocation of animals to the different treatment groups (five to eight animals per group) was performed randomly. The different number of animals per experimental groups is a result of multiple experiments or deaths that occurred before experimental end points.

Endotoxemia. Mice were injected intraperitoneally with lipopolysaccharide (LPS; Escherichia coli 0111:B4; Sigma) dissolved in sterile, pyrogen-free saline that was sonicated for 30 mins immediately before use. Two hours after injection, blood was collected, allowed to clot for 2 hrs at room temperature, and then centrifuged at room temperature for 15 mins at 2000×g. Serum samples were stored at −20° C. before analysis. Enzyme-linked immunosorbent assay was used to measure tumor necrosis factor (TNF) concentrations according to the manufacturer's instructions (R&D Systems, Minneapolis, Minn.).

Electrical Vagus Nerve Stimulation. Mice were anesthetized by isoflurane (1.5% to 2.5%) using a cone mask. A midline cervical incision was made to expose the left vagus, which was suspended across bipolar platinum electrodes (Plastics One) connected to a stimulation module (STM100C, Biopac Systems) and controlled by an acquisition system (MP150, Biopac Systems). Electrical stimulation variables were programmed using AcqKnowledge software (Biopac Systems) and included 1 V, 2 milliseconds, 5 Hz for 20 mins (10 mins before LPS and 10 mins after); 1 V, 2 milliseconds, 5 Hz for 2 mins (1 min before LPS and 1 min after); 1 V, 2 milliseconds, 5 Hz for 30 seconds (5 mins after LPS); and 1 V, 0.5 milliseconds, 30 Hz for 30 seconds (5 mins after LPS). Sham-operated mice underwent subcutaneous tissue dissection without exposure of the vagus nerve.

Subdiaphragmatic Vagotomy. A midline laparotomy incision was made, and the intestines and stomach were retracted to expose the distal esophagus. The ventral vagal branch was identified and divided using sharp dissection. The esophagus was encircled and rotated to expose and divide the dorsal vagal branch. The incision was closed using 6-0 polypropylene sutures. Animals received 1 mL of 0.9% normal saline subcutaneously after surgery. Mice recovered for 7 days before further experimentation.

Heart Rate Measurement. Two unipolar electrodes were placed onto the anterior chest wall of anesthetized mice and attached to an amplifier (ECG100C) and processing unit (MP150). The electrocardiogram was processed offline using AcqKnowledge software. Baseline electrocardiograms were recorded for 5 mins. Experimental groups included neck dissection (sham surgery), vagus nerve dissection, transcutaneous vagus nerve stimulation, or escalating doses of electrical vagus nerve stimulation (1 V, 5 Hz, 2 milliseconds; 5 V, 5 Hz, 2 milliseconds; 5 V, 30 Hz, 0.5 milliseconds). Electrocardiograms were recorded and mean heart rates were compared with baseline values.

Generation of Primary Human Macrophages. Peripheral blood mononuclear cells were isolated from the blood of normal volunteers (Long Island Blood Services, Melville, N.Y.) over a Ficoll-Hypaque (Pharmacia Biotech, Uppsala, Sweden) density gradient. Monocytes were isolated by adherence. Macrophages were generated with 2 ng/mL human MCSF for 5 days in RPMI 1640 medium (Life Technologies, Grand Island, N.Y.) supplemented with 10% human serum, penicillin (100 units/mL, Life Technologies), streptomycin (100 μg/mL, Life Technologies), and glutamine (2 mM, Life Technologies). To determine the effect of acetylcholine on endotoxin response, the cells were treated with acetylcholine chloride (10 μM, Sigma) in the presence of acetylcholine esterase inhibitor (pyridostigmine bromide, 1 mM) in Opti-MEM I media (GibcoBRL) for 1 hr. The cells were washed once with 1× phosphate buffered saline (Fisher) and suspended in complete medium supplemented with human MCSF. The macrophages received 4 hrs of LPS (10 ng/mL, E. coli 0111:B4, Sigma) stimulation in Opti-MEM I, beginning 2, 24, 48, and 72 hrs after acetylcholine exposure. Supernatants were collected and TNF was analyzed by enzyme-linked immunosorbent assay.

Cecal Ligation and Puncture. CLP was performed on BALB/c mice as described previously (19). Animals received antibiotic (Primaxin; 0.5 mg/kg, subcutaneously) and 0.9% normal saline (20 mL/kg body weight, subcutaneously) immediately after surgery. Mice underwent transcutaneous vagus nerve stimulation or sham stimulation beginning 24 hrs after surgery and thereafter twice a day for 2 days. Serum HMGB1 levels were determined 44 hrs after surgery by Western blot as described previously (14, 17). HMGB1 concentrations were calculated against standard curves generated using purified recombinant HMGB1 (14, 17). Serum interleukin-10 levels were measured using Cytometric Bead Array (Becton Dickinson) according to the manufacturer's instructions.

Clinical Sickness Score. Animals were assessed 44 hrs after CLP. The sickness score is composed of four clinical signs, including diarrhea, piloerection, spontaneous eye opening, and activity level. The presence of each component is worth either 1 point (diarrhea, piloerection) or 1-2 points (eye opening, activity level), depending on severity. A maximum score of 6 denotes a moribund animal with 100% mortality within 24 hrs. A score of 0 denotes a healthy, nonseptic animal.

Statistics. All data are expressed as mean±SEM. One-way analysis of variance followed by the Bonferroni correction was used to compare mean values between three groups. The two-tailed Student's t-test was used to compare mean values between two groups. Differences in survival were determined using the log-rank test. P values <0.05 were considered significant.

Results

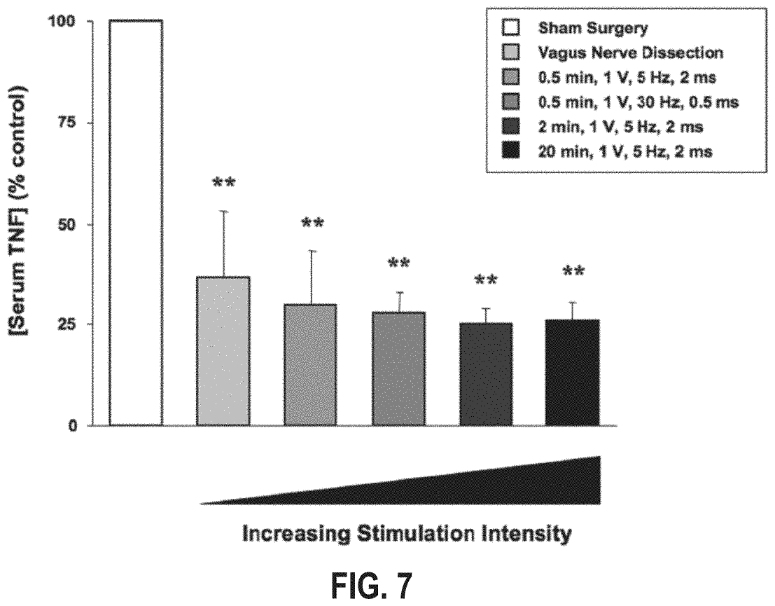

Electrical stimulation was chronically administered to mice using implanted wire electrodes. Increasing doses of electricity following surgical isolation of the vagus nerve did not suppress TNF concentrations further, as illustrated in FIG. 7.

Anti-Inflammatory and Cardioinhibitory Effects of Vagus Nerve Stimulation are Dissociable.

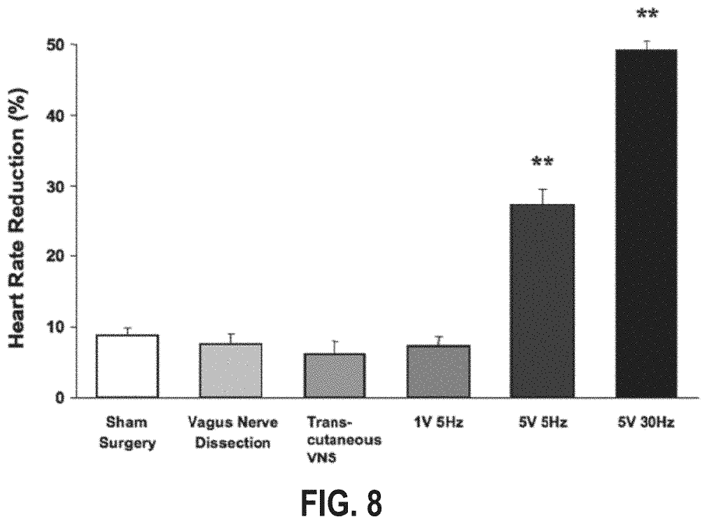

The vagus nerve modulates numerous autonomic functions (20). To address the specificity of vagal anti-inflammatory signaling, we measured heart rate in animals receiving vagus nerve dissection, transcutaneous vagus nerve stimulation, or electrical vagus nerve stimulation (1 V, 5 Hz, 2 milliseconds) and observed no significant effects on heart rate with any of these treatments, as shown in FIG. 8. To ensure that vagus nerve stimulation can elicit “classic” cardioinhibitory effects, we delivered more intense stimuli (5 V, 5 Hz, 2 milliseconds; or 5 V, 30 Hz, 2 milliseconds) and observed a 27.3%±2.3% and 49.1%±2.1% decrease in heart rate, respectively (p<0.05).

Effective Duration of Action of Vagus Nerve Stimulation and Cholinergic Agonists is Between 48 and 72 Hrs.

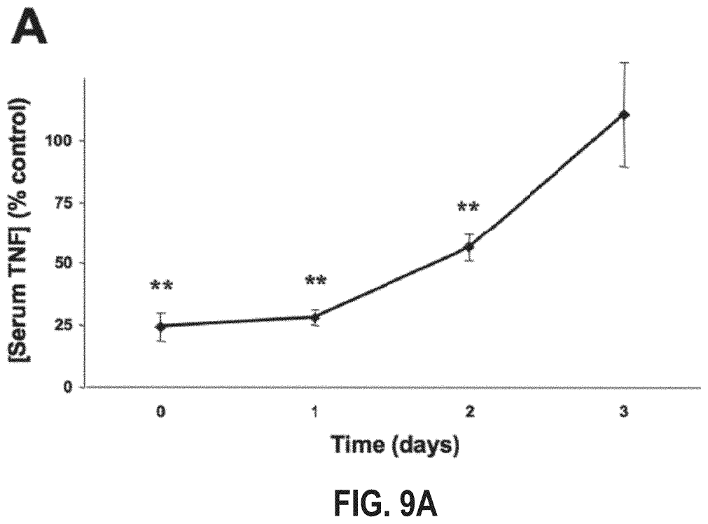

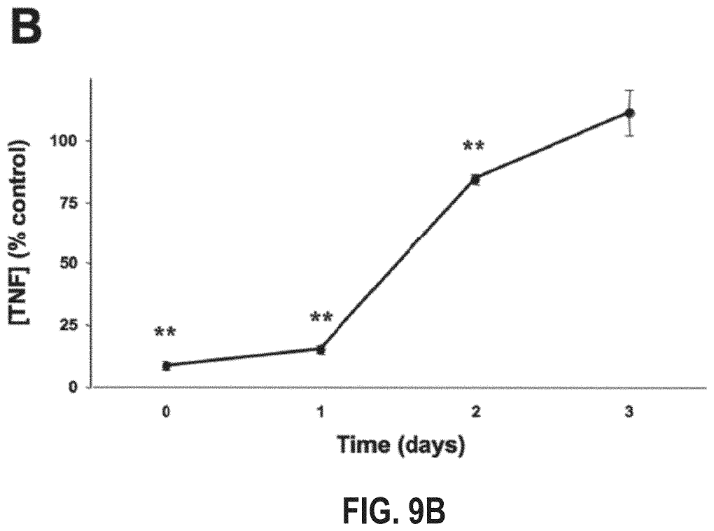

To study the effective duration of action of vagal anti-inflammatory signaling, we gave the mice electrical vagus nerve stimulation or sham stimulation, followed by defined recovery periods before endotoxin challenge. We observed that vagus nerve stimulation significantly reduced serum TNF concentrations when LPS was administered 48 hrs following treatment, but not after 72 hrs. This is illustrated in FIG. 9A.

To elucidate a mechanism for this relatively long neural duration of action of the anti-inflammatory effect of vagus nerve stimulation, we next examined the TNF-suppressive effects of cholinergic agonists on endotoxin-stimulated primary human macrophages. Macrophage cell cultures were stimulated with acetylcholine together with the acetylcholinesterase inhibitor pyridostigmine for 1 hr, and then cells were washed and suspended in fresh media. Macrophages were stimulated with endotoxin 24, 48, or 72 hrs postexposure to cholinergic treatment. We observed that cholinergic treatment significantly attenuated TNF release for 48 hrs, but not 72 hrs after exposure, as shown in FIG. 9B. These results suggest that cholinergic signaling in macrophages may contribute to the long-lived anti-inflammatory effects of vagus nerve stimulation. For example, refer to FIG. 9A.

Vagus Nerve Stimulation Fails to Induce Hyperglycemia or Cell Death.

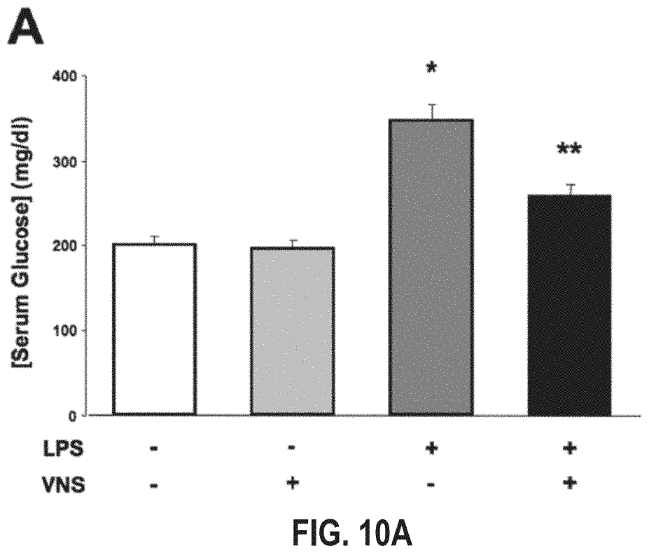

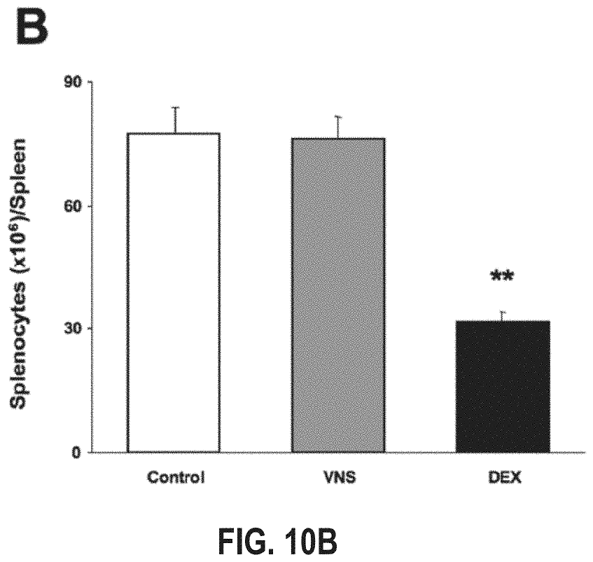

The systemic side effect profile of vagus nerve stimulation is currently unknown. We compared vagus nerve stimulation to administration of glucocorticoids, a previously known approach in the treatment of inflammatory diseases, by measuring serum glucose concentrations and splenocyte viability in endotoxemic mice, as shown in FIG. 10A. We observed that vagus nerve stimulation significantly lowered serum glucose concentrations during lethal endotoxemia. Administration of dexamethasone significantly reduced viable splenocyte counts, while vagus nerve stimulation preserved splenocyte viability, as illustrated in FIG. 10B.

As discussed above, the nervous system regulates pro-inflammatory cytokine production through an α7nAChR-dependent, vagus nerve mediated pathway to the spleen. Electrical vagus nerve stimulation inhibits pro-inflammatory cytokine production and prevents lethal shock in acute models of systemic inflammation.

HMGB1 is a necessary and sufficient mediator of lethal organ damage in murine CLP sepsis. Systemic HMGB1 concentrations are significantly elevated in this model, while neutralizing antibodies directed against HMGB1 significantly reduce organ damage and improve survival. Pharmacologic agents that reduce circulating HMGB1 concentrations, such as ethyl pyruvate and nicotine, also provide significant protection against polymicrobial sepsis lethality. In addition, infusion of HMGB1 to rodents causes organ damage and epithelial barrier failure. On the contrary, the pluripotent, pro-inflammatory cytokine TNF has not been found to be a critical mediator of organ damage and lethality in this model. Vagus nerve stimulation significantly reduces systemic HMGB1 concentrations in septic mice and thereby protects against the development of lethal organ damage.

Pharmacologic α7nAChR agonists, such as nicotine, can mimic the effects of vagus nerve stimulation but may be complicated by poor specificity and systemic toxicity. We show here that vagal anti-inflammatory signaling is specific and dissociable from heart rate regulation, suggesting that it may be a less toxic route for activating the cholinergic anti-inflammatory pathway. Interestingly, the vagus nerve is comprised of A, B, and C fiber subtypes, and the B and C subtypes have been implicated in mammalian heart rate regulation. The dissociability and lower activation threshold of vagal anti-inflammatory signaling allow us to hypothesize that the A fibers, which have the lowest activation threshold and do not appear to participate in heart rate regulation, may fulfill the role of cholinergic anti-inflammatory fibers.

We also showed that vagus nerve stimulation suppressed serum TNF levels even when applied 48 hrs before LPS administration, which is comparable to the cholinergic suppression of TNF in vitro. Future studies will contribute to revealing detailed mechanisms underlying this long-lasting anti-inflammatory efficacy. Current anti-inflammatory therapies, such as glucocorticoids, are associated with serious side effects, including hyperglycemia and immune cell apoptosis. We found that vagus nerve stimulation does not reduce splenocyte viability, while glucocorticoids significantly deplete viable splenocytes, suggesting that vagus nerve stimulation may be less toxic to the spleen. Vagus nerve stimulation also significantly lowers serum glucose levels during systemic inflammation. Recent studies have demonstrated the adverse effects of hyperglycemia in critically ill patients and the improvement in outcome that results from tighter glycemic control.

The description above may be readily generalized to describe various parameters and effects for vagus nerve stimulation that are new and very unexpected. In particular, the extremely low level of stimulation (both low intensity, and low duty-cycle) have been seen to have a profound—and chronic—effect on the immune response. By low duty-cycle effect, we are referring to the time stimulation is “on” compared to the time stimulation is “off” (e.g., the ratio of “on” to “off”). Thus, the stimulation (and particularly chronic stimulation) described herein is “off” much more that in it is “on”. The high efficacy of these low duty-cycle modulation parameters is unexpected, and may have a profound effect on the way that treatment of immune response (humor and/or cellular) are performed. For example, chronic (or long-term) stimulation may be effectively performed without the risk of negative effects associated with known stimulation protocols of the vagus nerve.

Furthermore, the low-intensity (and low duty cycle) situation may be used safely without interfering with other aspects controlled by the vagus nerve. For example, altered heart rate and blood pressure are not seen with the low-intensity, low duty-cycle modality stimulation. Thus, the vagus nerve may be stimulated either mechanically or electrically in a low-intensity and low duty-cycle fashion without desensitizing the vagus nerve or its targets, and without invoking a potentially unwanted physiological effect (e.g., on heart rate, heart tone, blood pressure, gastric processes, or any other effect attributed to vagal nerve stimulation except the targeted immune response).

Finally, the disclosure here may be used to support mechanical stimulation and electrical stimulation modalities. For example, implantable mechanical and implantable electrical stimulators may be used having low-intensity and low duty-cycle stimulation protocols, as described. Stimulation of the vagus nerve may be performed externally or internally.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating an inflammation in a patient, the method comprising:
applying electrical stimulating from an implanted neurostimulator to a nerve of the cholinergic anti-inflammatory pathway to inhibit pro-inflammatory cytokine production, wherein a controller of the implanted neurostimulator limits the output of the implanted neurostimulator to apply 1.8 coulombs or less every two hours.

2. The method of claim 1, wherein the controller of the implanted neurostimulator limits the output of the implanted neurostimulator to 180 millicoulombs or less every two hours.

3. The method of claim 1, wherein the controller of the implanted neurostimulator limits the output of the implanted neurostimulator to 90 millicoulombs or less every two hours.

4. The method of claim 1 wherein an off-time before the nerve of the cholinergic anti-inflammatory pathway is re-stimulated is from 2 hours to 24 hours.

5. The method of claim 1, wherein an off-time before the nerve of the cholinergic anti-inflammatory pathway is re-stimulated is 4 hours or more.

6. The method of claim 1, wherein the method is a method of treating rheumatoid arthritis.

7. The method of claim 1, further comprising monitoring the effect of the stimulation on inflammation in the patient.

8. The method of claim 1, further comprising determining the level of a pro-inflammatory cytokine prior to stimulation.

9. The method of claim 1, wherein applying electrical stimulating from the implanted neurostimulator to the nerve of the cholinergic anti-inflammatory pathway comprises applying electrical stimulation to one or more of: a vagus nerve, a splenic nerve, a hepatic nerve, a facial nerve, and a trigeminal nerve.

10. A method of treating rheumatoid arthritis in a patient, the method comprising:
applying electrical stimulating from an implanted neurostimulator a nerve of the cholinergic anti-inflammatory pathway to inhibit inflammation, wherein a controller of the implanted neurostimulator limits the output of the implanted neurostimulator to apply 1.8 coulombs or less every two hours.

11. The method of claim 10, wherein the controller of the implanted neurostimulator limits the output of the implanted neurostimulator to 180 millicoulombs or less every two hours.

12. The method of claim 10, wherein the controller of the implanted neurostimulator limits the output of the implanted neurostimulator to 90 millicoulombs or less every two hours.

13. The method of claim 10, wherein an off-time before the nerve of the cholinergic anti-inflammatory pathway is re-stimulated is from 2 hours to 24 hours.

14. The method of claim 10, wherein an off-time before the nerve of the cholinergic anti-inflammatory pathway is re-stimulated is 4 hours or more.

15. The method of claim 10, further comprising monitoring the effect of the stimulation on inflammation in the patient.

16. The method of claim 10, further comprising determining the level of a pro-inflammatory cytokine prior to stimulation.

17. A method of treating a chronic inflammatory disorder in a patient, the method comprising:
applying electrical stimulating from an implanted neurostimulator a nerve of the cholinergic anti-inflammatory pathway to inhibit inflammation, wherein a controller of the implanted neurostimulator limits the output of the implanted neurostimulator to apply 900 millicoulombs or less every two hours.

18. The method of claim 17, wherein the chronic inflammatory disorder is selected from the group consisting of appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease.

19. The method of claim 17 wherein an off-time before the nerve of the cholinergic anti-inflammatory pathway is re-stimulated is from 2 hours to 24 hours.

20. The method of claim 17, wherein the off-time before the nerve of the cholinergic anti-inflammatory pathway is re-stimulated is between about 16 and about 30 hours.
